(19) United States
(12) Patent Application Publication
(10) Pub. No.: US 2026/0287552 A1
(43) Pub. Date: Sep. 24, 2026

(54) System and Method for the Rapid Determination of Clean Fill Materials Associated With Excess Excavated Materials

(71) Applicant: Thunderbird Environmental, LLC, Clinton, CT (US)

(72) Inventor: Darby Wayne Hittle, Clinton, CT (US)

(73) Assignee: Thunderbird Environmental, LLC, Clinton, CT (US)

(21) Appl. No.: 19/085,044

(22) Filed: Mar. 20, 2025

(51) Int. Cl. G01N 27/64; G01N 33/24

(52) U.S. Cl. CPC G01N 27/64; G01N 33/24

(57) ABSTRACT

A method for the determination of whether excess excavated material can be considered to be clean fill includes the steps of: receiving a load of soil removed from a location and taking a representative sample thereof, performing a location-based analysis of the soil based at least in part on the location from which the soil was removed, performing a series of physical observations of the soil, performing field screening tests on the representative sample of the soil, and making an initial determination that the soil is clean fill if results of the location-based analysis, results of the series of physical observations and results of the field screening tests are all indicative of no contamination. The load of soil is then combined with other loads of soil, their representative samples are combined, and further laboratory analysis is performed, as appropriate to arrive at a final clean fill determination.

### FIELD OF THE INVENTION

This disclosure relates to a system and method for the rapid determination whether excess soil and spoils, for example generated during utility trenching, road work, and other site development activities (excess excavated materials), can be considered clean fill.

### BACKGROUND OF THE INVENTION

Polluted soil is regulated in many states pursuant to various governmental regulations. In some of these states, polluted soil that is excavated from known release sites must be characterized to determine whether it can be reused as “clean fill,” as again defined by various governmental solid waste regulations. Many licensed environmental professionals take the position that subjecting excess excavated materials to a broad range of laboratory analysis, from any site in such regulated states, is required to prove such materials are not polluted soil that can be reused as clean fill, which is a time-consuming, costly, and unnecessary approach. Also, subjecting excess excavated material to a broad range of laboratory analysis without proper rationale often yields laboratory detections of compounds, such as polynuclear aromatic hydrocarbons and metals which could reasonably be attributed solely to the presence of clean asphalt paving materials (which is often considered clean fill by regulation) and natural soil when there is no other evidence to suggest that the material has been impacted by the release of a substance. However, failure to perform any sort of evaluation of excess excavated material prior to reuse can result in the contamination of receiving sites, if there were previously unknown spills that impacted the material. Both approaches can be problematic.

Various governmental solid waste regulations, as applied to excess soil/spoils, differentiate between “solid waste” and “clean fill.” “Solid waste” can be defined as meaning “unwanted or discarded solid, liquid, semisolid, or contained gaseous material, including, but not limited to, demolition debris, material burned or otherwise processed at a resource's recovery facility or incinerator, material processed at a recycling facility and sludges or other residues from a water pollution abatement facility, water supply treatment plant or air pollution control facility.” “Disposal” or placement of solid waste generally requires a permit or other regulatory authorization.

In contrast, “clean fill” can be defined as follows:
(1) natural soil;
(2) rock, brick, ceramics, concrete, and asphalt paving fragments (which are virtually inert and pose neither a pollution threat to ground or surface waters nor a fire hazard); and
(3) polluted soil that has been treated to reduce the concentration of pollutants to levels which do not exceed applicable governmental regulations.

To reiterate, there are three types of material that can be clean fill: (1) natural soil, (2) rocks, brick, ceramics, concrete, and asphalt paving fragments, and (3) polluted soil that has been appropriately treated. By definition, only polluted soil requires sampling and laboratory analysis to determine whether it is suitable for reuse. If excess excavated material can be determined to meet either of the first two components of the definition of clean fill, there is no need for expensive and time-consuming, exhaustive laboratory analysis in order to reuse it. Therefore, it is critical to determine whether excess excavated material meets the first two possible definitions of clean fill: 1) natural soil, or 2) rocks, brick, ceramics, concrete, and asphalt paving fragments.

Thus, the question is as follows: should entities generating excess soils characterize all excavated materials by subjecting the materials to a broad range of laboratory analysis in an attempt to demonstrate compliance with regulations that may or may not be applicable to the materials? The present invention has the potential to reduce final excess excavated material disposition costs significantly, by providing a system and method of rapid clean fill determination that avoids improper designation of clean fill materials such as asphalt paving materials and natural soil materials as polluted soil.

However, a failure to perform any characterization of the excess excavated materials could present environmental liability risk resulting from the reuse of such materials. Thus, due diligence is required to predetermine (without testing) whether the excess excavated materials have the potential to have been impacted by a release. If there is evidence of a release (either from predetermination or observed while in the field), then excess excavated materials must be treated as polluted soil that is not clean fill. The presumption of the inventive rapid clean fill determination (RCFD) system and method according to the present invention is that if there is no evidence of a historical or potential release at the location of the excess excavated material, then proceed as if such material consists of natural soil or “rocks, brick, ceramics, concrete, and asphalt paving fragments”. There remains a need, however, to confirm this presumption through representative sampling and selective laboratory analysis before concluding the material is clean fill.

Additionally, unintentional/inadvertent generation of “polluted soil” often occurs when facilities subject excess excavated materials to a broad range of laboratory analysis, often unnecessarily. Subjecting excess excavated material to a broad range of laboratory analysis without proper rationale often yields laboratory detections of compounds such as polynuclear aromatic hydrocarbons and metals which could reasonably be attributed solely to the presence of asphalt paving materials (which is often considered clean fill by regulation) and natural soil when there is no other evidence that the material has been impacted by the release of a substance. Therefore, by implementing the RCFD system and method as described herein, soil management facilities and generators (the person, organization, or entity responsible for managing the excess excavated materials) can avoid improper designation of clean fill materials (natural soil, clean brick, concrete and asphalt paving materials) as polluted soil.

Lastly, the RCFD system and method provides soil management facilities with a much needed option for excess soil/spoils screening/characterization that has the potential to yield clean fill. Clean fill can typically be utilized in construction projects without the usual regulatory restrictions and requirements typically associated with reuse of “polluted soil.” Often, polluted soil that cannot be utilized in some form of beneficial reuse requires transportation and off-site disposal at an approved/licensed disposal facility at great expense. While there are costs associated with disposal of excess clean fill materials which have no apparent use, disposal costs associated with disposition of clean fill materials vs. polluted soil can be significantly lower.

The present invention, therefore, aims to remedy the problems associated with currently practiced excess excavated materials management techniques by balancing the need for due diligence with the desire to ensure that costs associated with reuse of excess excavated materials are minimized by avoiding overly aggressive laboratory analysis when such is not necessary.

### SUMMARY OF THE INVENTION

The present invention achieves these objectives by providing a method for the determination of whether excess excavated material can be considered to be clean fill, the method including the steps of: receiving a load of soil removed from a location and taking a representative sample thereof, performing a location-based analysis of the soil based at least in part on the location from which the soil was removed, performing a series of physical observations of the soil, performing field screening tests on the representative sample of the soil, and making an initial determination that the soil is clean fill if results of the location-based analysis, results of the series of physical observations and results of the field screening tests are all indicative of no contamination.

If the initial determination is that the soil is clean fill, then the method further includes the steps of combining the load of soil with other loads of soil that have also been initially determined to be clean fill to define a composite load, combining the representative samples of all loads of soil that have been combined to define a composite sample, subjecting the composite sample to laboratory analysis, and making a final determination that all of the loads of soil defining the composite load are clean fill if results of the laboratory analysis fall within acceptable parameters.

In some embodiments, the step of performing a location-based analysis comprises the step of comparing the location from which the soil was removed to at least one public environmental record of known or potentially contaminated sites. In some embodiments, the step of performing a location-based analysis comprises the step of determining whether there have been any in-field observations indicative of a potential release at the location. In certain of these embodiments, the step of determining whether there have been any in-field observations indicative of a potential release at the location comprises the step of interviewing a driver of a truck used to transport the load of soil.

In some embodiments, the series of physical observations comprise at least one of visual observations and olfactory observations. In certain of these embodiments, the series of physical observations comprise visual observations and the visual observations are considered to be indicative of contamination if staining and/or debris is observed. In certain embodiments, the series of physical observations comprise olfactory observations and the olfactory observations are considered to be indicative of contamination if chemical, petroleum or otherwise offensive odors are observed. In some embodiments, the series of physical observations are performed by at least two different persons.

In some embodiments, the field screening tests employ a photoionization detector. In certain of these embodiments, the field screening tests are considered to be indicative of contamination if the photoionization detector indicates a reading of greater than 100 ppm.

In some embodiments, the laboratory analysis comprises tests for at least the following: polychlorinated biphenyls (PCBs), arsenic, barium, cadmium, chromium, lead, mercury, selenium and silver (total RCRA 8 metals).

In some embodiments, the method further includes the step of making a determination that the soil comprises contaminated materials if results of at least one of the location-based analysis, the series of physical observations and the field screening tests are indicative of contamination.

In some embodiments, if the initial determination is that the soil comprises clean fill, then the method further includes the step of making a final determination that all of the loads of soil defining the composite load comprise contaminated materials if results of the laboratory analysis fall outside acceptable parameters.

In accordance with another aspect of the present invention, a method for the determination of whether excess excavated material can be considered to comprise clean fill includes the steps of receiving a load of soil removed from a location and taking a representative sample thereof, performing a location-based analysis of the soil based at least in part on the location from which the soil was removed by comparing the location from which the soil was removed to at least one public environmental record of known or potentially contaminated sites and by determining whether there have been any in-field observations indicative of a potential release at the location, performing a series of physical observations of the soil, the series of physical observations including at least one of visual observations and olfactory observations, employing a photoionization detector to perform field screening tests on the representative sample of the soil, and making an initial determination that the soil comprises clean fill if results of the location-based analysis, results of the series of physical observations and results of the field screening tests are all indicative of no contamination.

If the initial determination is that the soil comprises clean fill, then the method further includes the steps of combining the load of soil with other loads of soil that have also been initially determined to be clean fill to define a composite load, combining the representative samples of all loads of soil that have been combined to define a composite sample, subjecting the composite sample to laboratory analysis, and making a final determination that all of the loads of soil defining the composite load comprise clean fill if results of the laboratory analysis fall within acceptable parameters.

In some embodiments, the step of determining whether there have been any in-field observations indicative of a potential release at the location comprises the step of interviewing a driver of a truck used to transport the load of soil.

In some embodiments, the series of physical observations comprise visual observations and the visual observations are considered to be indicative of contamination if staining and/or debris is observed. In some embodiments, the series of physical observations comprise olfactory observations and the olfactory observations are considered to be indicative of contamination if chemical, petroleum or otherwise offensive odors are observed. In some embodiments, the series of physical observations are performed by at least two different persons.

In some embodiments, the field screening tests are considered to be indicative of contamination if the photoionization detector indicates a reading of greater than 100 ppm.

In some embodiments, the laboratory analysis comprises tests for at least the following: polychlorinated biphenyls (PCBs), arsenic, barium, cadmium, chromium, lead, mercury, selenium and silver (total RCRA 8 metals).

In some embodiments, the method further includes the step of making a determination that the soil comprises contaminated materials if results of at least one of the location-based analysis, the series of physical observations and the field screening tests are indicative of contamination.

In some embodiments, if the initial determination is that the soil comprises clean fill, then the method further includes the step of making a final determination that all of the loads of soil defining the composite load comprise contaminated materials if results of the laboratory analysis fall outside acceptable parameters.

The present invention, as set out above in various respects, provides a soil management program for the characterization of excess soil as clean fill versus not clean fill, so as to balance the need for due diligence with the requirement to ensure that costs are kept reasonable by avoiding overly aggressive laboratory analysis when such is not necessary, and often leads to improper designation of clean fill materials such as asphalt paving materials and natural soil materials as polluted soil.

Other features and advantages of the invention will become more apparent from consideration of the following drawings and written description.

### BRIEF DESCRIPTION OF THE DRAWINGS

FIG. 1 is a schematic representation depicting a correlation of soil colors with the presence of various substances;

FIG. 2 is a chart setting forth various standard accepted olfactory observations; and

FIG. 3 is a flow chart illustrating at a high level an exemplary method for the determination of whether excess excavated material can be considered clean fill in accordance with the present invention.

### DETAILED DESCRIPTION OF THE INVENTION

The present invention may be further understood with reference to the following description and the appended drawings, wherein like elements are referred to with the same reference numerals.

Excess soil and spoils (excess excavated materials) are generated during utility trenching, road work, routine municipal maintenance and improvements, and roadway repairs, and other site development activities. The excess excavated materials are transported via truck to a staffed facility that includes: an office, truck scale, multiple soil material bins, and heavy equipment to move and load large quantities of soil. Facility staff include at least one environmental scientist who at a minimum possesses a 4-year bachelor's degree from an accredited university in either geology, environmental science, engineering, or a related field of study and who is being overseen by a licensed environmental professional (LEP). The facility staff responsible for implementing the inventive process uses several techniques and criteria to determine whether such excess excavated materials comprise clean fill, as follows:
(1) Truck Driver Interview Information: the truck driver is interviewed to confirm source location (address/location where material was excavated and loaded onto the truck or roll-off container) and whether the driver noted any indications that the material being delivered is not clean fill (e.g., notable odors or visible staining) while the excess excavated material was being loaded onto the truck.
(2) Source Considerations: Only known (previously identified project and location) inbound trucks are accepted. Source location is compared to public environmental records. The results of the foregoing comparison are used to initially determine whether the materials are likely clean fill or not.
(3) Observation (Visual and Olfactory): The inbound load is tipped and then observed. The facility personnel also perform visual and olfactory observations.
(4) Field Screening: The load is field screened using a composite (e.g., five-point) sample in an enclosed bag with headspace for volatile organic chemical vapors using a photoionization detector (PID).
(5) Preliminary Clean Fill Determination: Using the observational information and field screening data, the facility personnel preliminarily determine whether or not the soils constitute clean fill.
(6) Bin Composite Sample Collection: Before moving to a bin (see below), the above referenced 5-point composite sample of the truckload of excess excavated material is taken and stored in a container consistent with the storage bin.
(7) Bin Storage: Following preliminary determination as to whether or not the excavated material comprises clean fill, the material moved to a storage bin designated as “Clean Fill” or “Presumed Contaminated Material (PCM).”
(8) Analytical Clearance Check and Final Clean Fill Determination: Once a Clean Fill bin is full, the accumulated representative bin composite sample that was previously/continuously collected from each truck load of soil as the bin was being filled is sent to an environmental chemistry laboratory to provide specified chemical data. The material in the bin is confirmed to be clean fill, or not, based on the results of the laboratory analysis.

The inventive system and method provides a structured framework and specific tools that allow the above process to work effectively and responsibly. In the following sections, more detailed descriptions of the tools used in the process are provided, as is helpful guidance and hints for their use while implementing the inventive system and method.

### Source Considerations

Many state and municipal regulatory bodies maintain lists of contaminated or potentially contaminated properties and known releases (public environmental records). These records represent “hazardous waste facilities” and provide information regarding underground storage tanks, hazardous waste generators, sites listed on the National Priority List (Superfund), sites listed under the property transfer act, voluntary remediation sites, and more. Using these public environmental records, facility personnel can obtain a better sense of the nature of the source location, and whether the material coming from that address was potentially impacted by the release of a substance. While these records are not dispositive, they are an additional tool that helps facility personnel make conservative, informed decisions as to the nature of the excess excavated materials. These records are updated regularly; therefore, frequent checks are necessary to provide the best data to make informed decisions.

When a truck driver arrives and they have a specific street number associated with the address that is the source of the excess excavated material, facility personnel confirm that the address is for a known project location and the facility personnel search for the address in the public environmental records. If the materials were taken from the middle of a road or other non-addressed area and there is no specific house address that can be determined, the nearest street address or cross street location is used for searching the location.

In addition, facility personnel will also compare the source address to available state and local/municipal release area mapping resources, which can depict regionally known release areas.

If the source address is not on any public environmental record, then it is reasonable to infer that the source location of the excess excavated material is not known to be a site impacted by a release. Therefore, the load can be placed into a Clean Fill bin, assuming all other clean fill requirements have been met (as discussed further below).

### Visual Observations

To describe visual observations, the following characteristics are examined: the general color, texture, occurrence of large or small stones, debris/solid waste, organic debris such as tree limbs, wood, clean fill materials such as concrete, asphalt paving materials, and bricks, and other notable visual characteristics. Organic soils tend to be darker in color. If it is very dark, it could potentially contain organic material. If it was dug out near the surface of natural fill, it is very likely to contain humus (decomposed plant matter). To decide whether a soil is natural and undisturbed or altered by human activity, a soil's composition, color, and porosity is examined. Soil may have unnatural materials in it, such as glass, solid waste/trash and/or metal, while “natural soil” will not contain these items. Human-influenced fill tends to have a mottled color because it is composed of a mixture of different colored soil types.

In contrast, natural soil is comprised of: 1) humus, which is a dark organic matter, with live and decaying roots; 2) topsoil, which is comprised of a mixture of humus and leached minerals; and 3) subsoil, which is comprised of soil containing leached minerals like iron oxides and aluminum oxideslate. Leaching is the process of extracting substances from a solid by dissolving them in a liquid, which would be the natural groundwater in this instance. Below the subsoil is the parent material, which is where the weathered material originates; it is composed of partly weathered minerals and broken chunks of the bedrock below it, which is unweathered.

Four main factors influence the color of a soil:
(1) Mineral Matter—derived from the constituents of the parent material. Rocks are broken down to form soils, and sometimes these rocks give their color to the soil. More often, the color of the soil results from compounds, such as iron.
(2) Organic Matter—humus, the final stage of organic matter breakdown, is black. Throughout the stages of organic matter breakdown, the color imparted to the soil varies from brown to black. Sodium content influences the depth of color of organic matter and therefore the soil. Sodium causes the organic matter (humus) to disperse more readily and spread over the soil particles, making the soil look darker (blacker).
(3) Nature and Abundance of Iron Content—red, yellow, grey, and bluish-grey colors result from iron in various forms. Under average conditions of air and moisture, iron forms a yellow oxide imparting a yellow color to the soil. Where soils are well draining or under dry conditions, iron forms red oxides imparting a red color to the soil. Yet in waterlogged soil, with a lack of air, iron forms in a reduced state giving the soil grey/green/bluish-grey colors.
(4) Moisture Content—soil color darkens as the soil changes from dry to moist. But longer term color changes are linked to water relations as well. Careful observation of color can help to identify problems of waterlogging or leaching. Poorly drained soils are often dominated by blue-grey colors often with yellow mottling. Well-drained soils will usually have bright and uniform colors.

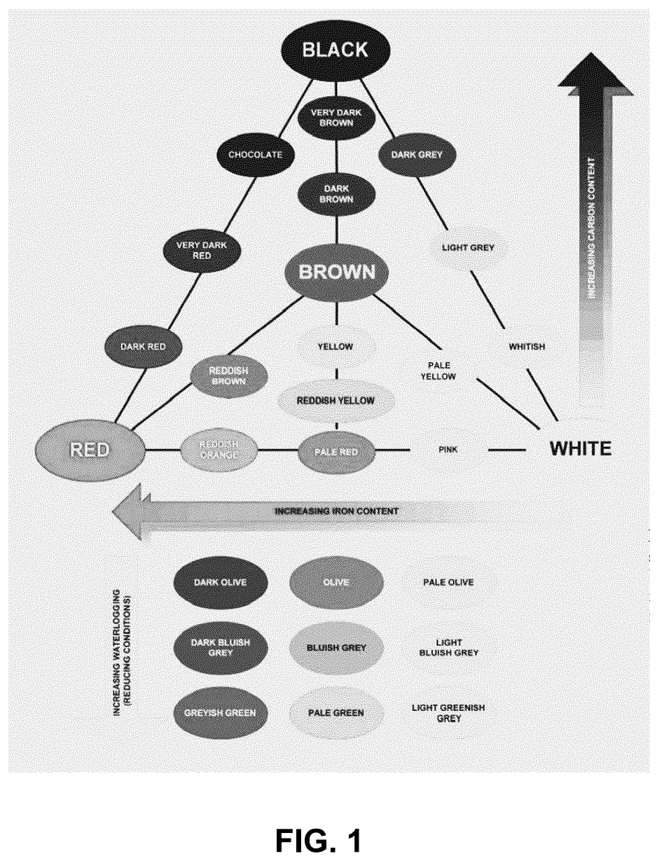

The soil color triangle chart reproduced in FIG. 1 illustrates relationships between color and environmental/chemical influencing conditions upon the color of soil.

As will be readily understood by those skilled in the art, this section is not an exhaustive explanation of soil description, which may be found in many soil science or geology books and the like. The purpose here is to provide general examples of guidance in describing observations of a tipped load of soil in terms of potential environmental contamination. These observations, together with olfactory observations and field screening, will serve as a basis for determining whether the materials are Clean Fill (as described more fully below).

### Olfactory Observations

Olfactory refers to the human sense of smell. Generally, when it comes to odors, they are characterized in terms of the following: character, intensity, frequency, and duration; however, for purposes of the present invention, an odor's character and intensity are the only characteristics of primary concern.

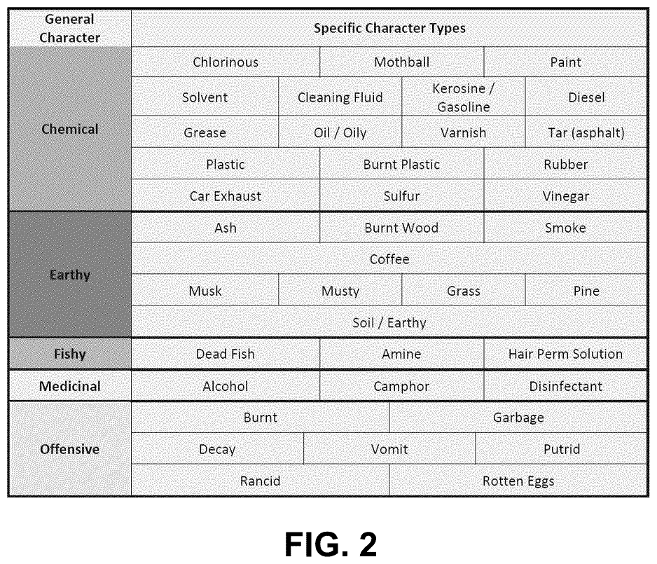

An odor's character is the actual description of what an odor “smells like.” This character is sometimes called the “quality” of the odor or the “offensiveness” of the odor. More offensive odors will be more annoying. Odor character is a basic scale of measurement where odors are characterized using reference vocabulary. Many different descriptor “wheels” have been developed for different businesses (beer, food, perfume, etc.) where the wheel is organized with general descriptors at the center and more specific characters listed towards the wheel rim. Reproduced in FIG. 2 is a table setting out some helpful odor reference vocabulary that may be useful in connection with characterizing soil samples.

An odor's intensity is the second building block and refers to the overall strength of the perceived odor. The more intense the odor, the more likely the odor will be annoying. Even pleasant odors (such as perfumes) can be very annoying at high intensities and, conversely, offensive odors can be very annoying at low intensities. Perceived odor intensity is the relative strength of the odor above the recognition threshold. Though there is no universally recognized scale for measuring odors, the following can be used to qualitatively assess odor strength for documentation purposes:
(1) Very Strong: Odor is evident on approach (beyond 6-10 feet or 2-3 meters from source) with or without cover.
(2) Strong: Odor is evident on approach (within 6-10 feet or 2-3 meters from source) without cover.
(3) Moderate: Odor is evident near the source with cover in place.
(4) Slight: Odor is evident near the source without a cover.
(5) None: No odor is evident, even near the source.

Odor evaluations are generally performed outdoors, where one has to cope with wind and odors from off-site sources. Such conditions may complicate observations and characterizations of odor character and intensity. It may be necessary to change location (move to the down-wind side of the pile). However, one should not put one's nose “into” or near the source; one should simply note the odor as the soil is approached and as the soil is handled to make a description of the color, texture, etc. It is preferable to make observations and describe them using a standardized set of descriptors, such as those shown in FIG. 2.

### Field Screening

After making visual and olfactory observations, a five-point composite of the tipped load is collected, typically using a soil knife. A small sample is taken from the north, east, west, south, and top sections of the soil pile produced by the tipped load. These sub-samples are placed into a plastic resealable bag and sealed closed. After allowing the sample to sit for five minutes, a screening is performed using a photoionization detector (PID).

PIDs detect and support monitoring of most volatile organic compounds (VOCs) with the best combination of speed of response, ease of use and maintenance, small size, and ability to detect low concentrations. These instruments use the ionization—i.e., the process that leads to the dissociation (separation) of a neutral (zero charge) atom or molecule into charged (positive or negative) particles, which are called ions—of a target compound as the basis of detection. When sampled, gas absorbs ultraviolet light (UV) energy from the PID lamp, which causes the gas to become excited, the result being an alteration of its molecular content, or ionization. UV energy knocks off an electron (e−) from the compound causing the compound to become a positively charged ion. Some substances are more easily ionized than others. The ionization potential (IP) is the measure of a substance's ability to be ionized, using an electron volt (eV) energy scale. This scale spans 7-16 eV. Substances with an IP of 7 eV are very easy to ionize and at 12-16 eV, they are difficult to ionize. Table 1 below shows the ionization potential of various substances that may be encountered.

TABLE 1
Ionization Potentials

| Substance | IP (eV) |
|---|---|
| Styrene | 8.47 |
| Xylene | 8.56 |
| Toluene | 8.82 |
| Vinyl Acetate | 9.19 |
| Benzene | 9.25 |
| Methyl Ethyl Ketone (MEK) | 9.51 |
| Acetone | 9.69 |
| Phosphine | 9.87 |
| Vinyl Chloride | 9.90 |
| Isopropyl Alcohol | 10.10 |
| Hexane | 10.13 |
| Propyl Alcohol | 10.22 |
| Ethyl Alcohol | 10.47 |

Chemicals ionized in a PID induce a current in the circuitry that displays the current in concentration units (PPM). A 10.6 eV lamp emits enough energy to ionize compounds with an IP rating of <10.6 eV. There are only a few compounds that require a stronger lamp. Thus, we have found that use of a 10.6 eV lamp is preferred. Ambient temperature must be above 32° F. and preferably above 50° F. Making measurements in high humidity or near electrical power sources (e.g., power lines and transformers) should be avoided.

PID response varies greatly with the nature of the VOCs encountered. Response is good for halogenated volatiles and aromatics but less so to alkanes. It should not be assumed that a low PID reading is sufficient evidence of no VOCs. PIDs typically have a linear range of detection between 1 to <300 PPM.

The Table 2 below shows the lower detection limit of exemplary substances, but conditions may impact the ability to measure, which are semi-qualitative at best.

TABLE 2
Lower Detection Limits

| Lower Detection | Ambient | Headspace | Headspace |
|---|---|---|---|
| Limit (ppm) | Air | Screening | Analysis |
| Gasoline: in Water | >100 | 10 s-100 s | 0.1-1.0 |
| Gasoline: in Soil | >100 | 10 s-100 s | 1.0-10 |
| Diesel: in Soil | >100 | 10 s-100 s | 10 s-100 s |

Action Threshold: For the inventive system and method, the threshold is considered to be 100 ppm, despite some states setting action thresholds at 25-50 ppm. Detections between 10-99 ppm may be actionable if correlated with visual and/or olfactory observations.

Based on observation and field screening, it is determined whether the load is designated as clean fill or not, as described more fully below. For those loads designated as clean fill, a scoop is then taken from the sample of the load using a 2 oz. soil scoop, sampling knife or the like and is added to a bin composite sample (for example collected in gallon-sized freezer bag or the like), which is then sealed. Collecting gravel or pieces of asphalt/bituminous is preferably avoided. The small soil bag may be discarded after the sample is taken from it. The bin composite sample bag is then placed into a corresponding plastic tote and a lid is secured thereon.

The Importance of Sampling Every Truck Load Received: The importance of collecting a representative composite sample of every load received (and not simply a composite sample of the full “bins” of material) cannot be overstated. Most (if not all) current soil management programs and soil stockpiling and staging facilities merely collect composite samples of materials received once a bin becomes full, and most (if not all) soil management programs and soil stockpiling and staging facilities do not collect a representative sample from every load of material which is received at the facility.

Composite samples collected from “full bins” of soil are often not sufficiently representative of the materials within the bins because the sampler (person who goes into the bin to collect the sample) typically collects composite samples from within the bins by walking on top of the material (a pile/full bin of soil which is typically 2-10 feet in height) and using hand sampling tools (spade/shovel) to collect shallow soil samples from the upper 6-12 inches of soil. Manual (hand) sampling full bins of soil at depths that would be sufficiently representative of the material within the bin is not typically possible due to the presence of boulders, concrete, asphalt paving fragments commonly found within the binned material. As such, these “full bin” composite samples are NOT sufficiently representative of the material which comprises the full bin of material for the purpose of determining whether the “binned” materials comprise clean fill.

Alternative “full bin” composite sampling methods could/would include vertical or horizontal drilling with mechanized soil coring equipment to ensure deeper (more representative) samples are collected from within the full bin of material. However, this “mechanized” sampling is rarely (if ever) utilized as a way to collect representative samples from full bins of soil material due to high costs, limited space in which to operate drilling and mechanized equipment within a bin structure, and obvious safety concerns. Additionally, there are currently no other known facilities or soil management programs that utilize the system and methods described herein for the rapid determination of clean fill materials. Known soil management facilities are believed to be conducting “binned material sampling” solely for the purposes of confirming possible recycling, reuse, or disposal options for accumulated “polluted soil”.

Typically, soil management facilities do not conduct composite sampling of “binned” materials for the purpose of making a “clean fill determination” as specified in the present invention. A typical soil management facility is less concerned with the importance of collecting bin samples that are sufficiently representative of the materials within the full bin, because these facilities to not plan to use the resulting sampling/analysis data for the purposes of making a clean fill determination. Excess excavated materials generated from utility excavations and road work often require: a) off-site disposal at an approved disposal facility; or b) some form of beneficial reuse of polluted soil when such permitted options are available.

Disposal and beneficial reuse options for excess excavated materials are largely dependent upon the results of analytical testing of representative samples. Soil disposal facilities and beneficial use permits typically specify analytical testing requirements for either disposal pre-authorization (disposal facilities), or compliance purposes (beneficial reuse permits).

When arranging for final disposition of excess excavated materials, and in consideration of cost and generator liability, soil management facilities often provide their clients (generators of the excess excavated materials) with multiple disposal/beneficial reuse options. As such, soil management facilities typically sample and analyze full-bins of material for a broad range of analytical parameters that may ultimately be needed for disposal or reuse. Some of the substances contained in clean fill (asphalt paving materials and natural soil) are among the analytes typically required as a pre-condition for disposal facility pre-approval or as a condition of a permitted beneficial reuse. Polyaromatic hydrocarbons (PAHs) and extractable total petroleum hydrocarbons (ETPH) are both common constituents of asphalt paving fragments, and naturally occurring metals, such as lead and arsenic, are common constituents of natural soil. However, often times, these substances (PAHs, ETPH, naturally occurring metals) which can be solely attributed to either the asphalt paving fragments and natural soil are often interpreted by environmental professionals and soil management facilities to conclude that the sampled material comprises polluted soil.

Soil management facilities that do not implement the RCFD system and method described in the present invention routinely (albeit inadvertently) create “polluted soil” by subjecting “binned” materials to a broad range of laboratory analysis which yields detections of compounds that can solely be attributed to the presence of asphalt paving materials (which is often considered clean fill by regulation) and natural soil. Typically, polluted soil that cannot be treated and utilized in some form beneficial reuse is transported to an approved/licensed disposal facility at great expense. As such, utilizing the present invention at soil management facilities has the potential to reduce final excess excavated material disposition costs significantly, by providing a system and method of rapid clean fill determination that avoids improper designation of clean fill materials such as asphalt paving materials and natural soil materials as polluted soil.

### Analytical Clearance Check

When it is concluded that a bin is full and is therefore closed, the representative bin composite sample (for that bin) that was previously/continuously collected from each truck load of soil as the bin was being filled representative composite sample of the bin's contents is collected and sent for laboratory analysis. The RCFD specified analytes for Clean Fill bin samples is presented below. Rapid sampling and turn-around of full bins ensure facilities are not overcrowded and remain available to accept more material. Upon sample preparation, the laboratory picks it up as soon as possible. A bin is full when the bin reaches its maximum recommended volume (i.e. 500 cubic yards of material).

The following is an exemplary procedure that may be employed for sample collection/preparation:
(1) Don appropriate personal protective equipment (PPE). It is important that facility personnel wear appropriate PPE before proceeding to collect a sample. The PPE includes safety glasses and clean nitrile gloves.
(2) Gather and decontaminate a stainless-steel bowl and sampling trowel. Care should be taken to ensure these items are completely clean and decontaminated before sample collection. This is necessary to avoid cross-contamination.
(3) Mark the bag(s) and tote with the close date and indicate the total number of bags used, e.g., “bag 1 of 1”, “bag 1 of 2”, “bag 2 of 2” etc.
(4) To create the sample, shake the bags (produced by five-point composites for each load while the bin was filling, see above) to blend and combine the material together, then add the material to the sampling bowl. If there is more material than the bowl can hold, dump a representative amount from each bag into the bowl. Leftover material will be used again if needed, therefore, do not discard.
(5) Gather all necessary sampling containers, as explained below.
(6) Transfer the material, with gloved hands, into the appropriate sampling containers. For soil jars, use a separate disposable plastic soil scoop for each sample to transfer material, filling each jar to the brim. For VOA vials, use a separate disposable soil core plunger for each sample, two plunges per vial.
(7) Take care to minimize the amount of non-soil material (i.e., rock, bituminous concrete, asphalt, organics, detritus [typically plant material but includes various kinds of solid waste]) to the extent possible. Non-soil materials may cause inaccurate, biased laboratory results.
(8) Ensure all container lids are tightly sealed, labeled appropriately, and placed on ice prior to scheduling a laboratory pickup. Complete a chain of custody (COC) for the samples and leave it with the cooler. A separate COC is required for each sample. Do not list samples from separate bins on the same COC. Request that the laboratory analyzes the samples on a three-day turnaround unless otherwise directed.
(9) When finished, add the remaining material from the sampling bowl back into the gallon sized bag to save, and place it back into the plastic tote until the results from the lab are received.
(10) Upon review of the laboratory results, if a re-test is necessary, then use the remaining soil in the gallon bag to make up another sample. If the lab results support the original soil bin determination as Clean Fill and no more samples are needed, then discard the remaining material and thoroughly clean the plastic tote for its next use.

The below Table 3 illustrates exemplary analyses to be performed and containers to be used for a Clean Fill material representative composite sample.

TABLE 3
Collection from Clean Fill Bins

| Soil Type | Analysis | Containers |
|---|---|---|
| Clean Fill | PCBs (ASE); total RCRA | 2x 8 oz Soil Jar |
| | 8 Metals; HVOCs*; TPH- | 2x low-level VOAs Vials |
| | GRO*; asbestos* | (DI water and/or stir bar) |
| | *as appropriate, | 1x high-level VOA Vial |
| | depending on source | (Methanol) |
| | location | |

The composite sample(s) are sent to a laboratory, which may perform several compound and elemental analyses, as indicated in Table 4 below together with interpretative guidance. At a minimum, testing is performed for PCBs and total RCRA 8 Metals & Mercury, with the other testing being optionally performed as appropriate, depending on source location.

TABLE 4
Possible Analyses Performed

| Analyte | Method | Interpretive Guidance |
|---|---|---|
| PCBs | 8282A | Any reported detections with a Laboratory |
| | | Reporting Limit of ≤0.1 mg/kg indicate that the |
| | | material has been impacted by a release of PCBs; |
| | | as such, the material does not comprise Clean Fill. |
| Total RCRA | 6010C | Reported detections which exceed metal-specific |
| 8 Metals & | 7471B | concentrations below indicate that the material has |
| Mercury | | been impacted by a release of metals; as such, the |
| | | material does not comprise Clean Fill: |
| | | Arsenic (As): 10 mg/kg |
| | | Barium (Ba): 4,000 mg/kg |
| | | Cadmium (Cd): 30 mg/kg |
| | | Chromium (Cr): 100 mg/kg |
| | | Lead (Pb): 100 mg/kg |
| | | Mercury (Hg): 10 mg/kg |
| | | Selenium (Se): 300 mg/kg |
| | | Silver (Ag): 300 mg/kg |
| Halogenated | 8260C | Any reported detections above the Laboratory |
| VOCs | | Reporting Limits indicate that the material has been |
| | | impacted by a release of halogenated VOCs; as |
| | | such, the material does not comprise Clean Fill. |
| TPH-GRO | 8015D | Any reported detections above the Laboratory |
| | | Reporting Limits indicate that the material has been |
| | | impacted by a release of TPH-GRO; as such, the |
| | | material does not comprise Clean Fill. |
| Asbestos | PLM | Any reported detections of asbestos above a |
| | EPA/600/ | concentration of 1% indicate that the material may |
| | R-93/116 | have been impacted by a release of asbestos, and |
| | (400 Point | supplemental analysis material does not comprise |
| | Count) | Clean Fill. |

As discussed above, initial determination (which is based on location but also on a series of screening observations) preliminarily determines whether materials within a bin comprise Clean Fill or not. Upon receipt of laboratory analysis results of the “full-bin” representative sample, a final determination is made of whether a bin of material comprises Clean Fill.

### Final Determination Methodology

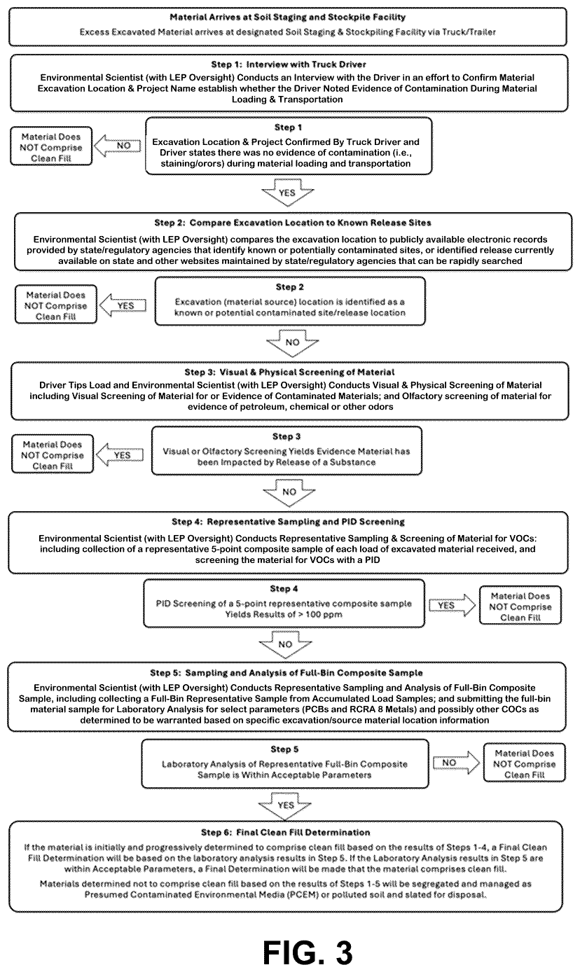

Using all of the information collected thus far, as described above, including the observational information and field screening data, the facility personnel, in consultation with a licensed environmental professional (LEP), make a final determination as to whether the excess excavated material comprises either clean fill or contaminated material. FIG. 3 is a flow chart outlining an exemplary the process used for initial, preliminarily/progress, and final clean fill determinations. As shown in FIG. 3, the inventive rapid clean fill determination (RCFD) system and method for the determination of whether of excess excavated material meets the definition of clean fill, can be considered as including the steps of:

Step 1: Interviewing the truck driver while receiving material in an effort to: a) confirm that the excess excavated material is being delivered by a known truck, from a known location, and from a known project; and b) establish whether the truck driver noted evidence of contaminated materials (odors, visible staining, or other) during loading & transportation of the excess excavated material.

Step 2: Comparing the location from which the material was excavated to publicly available records provided by state/regulatory agencies that identify known or potentially contaminated sites, or identified releases, including but not limited to leaking underground storage tanks.

Step 3: Visually/physically screening each load of excavated material in an effort to identify potential evidence of non-clean fill materials such as solid waste, urban fill materials (such as ash, burnt wood, or coal fragments), or staining.

Step 4: Collecting a representative 5-point composite sample from each load of material being received and physically screening the excess excavated material in the field using a Photoionization Detector (PID) for elevated levels of volatile organic compounds (VOCs) at concentrations greater than 100 parts per million (ppm).

If: (a) the interview with the truck driver yields no evidence to suggest that the material is not clean fill; (b) the source location is not identified as a potential or known contaminated site; (c) no evidence to suggest that the excess excavated material has been impacted by a release of a substance is identified during the initial visual screening or while collecting the representative composite sample; and (d) the results of the PID screening yields total VOC results of less than 100 ppm, then: the excess excavated materials are preliminarily determined to comprise clean fill. If the excess excavated material is not preliminarily determined to comprise clean fill, it is segregated from the clean fill material and designated for disposal at an approved/licensed disposal facility.

Before making a final clean fill determination and ultimately releasing materials preliminarily determined to be clean fill for reuse, laboratory analysis is performed as the next step in the RCFD method:

Step 5: A composite sample representative of the full bin of material (typically 500 cubic yards of material or more) will be submitted to a certified environmental laboratory to be analyzed for certain parameters. The “full-bin” material sample will be comprised of the cumulative/progressive 5-point composite samples (as previously described) representative of each of the individual loads of material which ultimately make up the full bin of material. As further described below, the analysis of a representative material sample that is representative of each of individual loads which make up the full bin is an essential component of the RCFD system and method. If the results of the laboratory analysis fall outside the acceptable parameters, that excess excavated material is segregated and designated for disposal at an approved/licensed disposal facility. However, if the results of the laboratory analysis fall within the acceptable parameters, a final step is performed as follows:

Step 6: if the material is initially, preliminarily, and progressively determined to comprise clean fill throughout Steps 1-5, a final clean fill determination is made, and the material is determined to comprise clean fill.

As described above, the RCFD method includes the following steps after receiving such material: (a) confirming the excess excavated materials is being received from a known location and source, and establishing whether the truck driver noted any evidence that the material has been impacted by a release of a substance; (b) determining whether, based on a review of public environmental records, there is reason to believe the source location of the material could be impacted by a release, (c) performing a series of physical observations of the excavated material; and (d) performing field screening tests on the excavated material; and (e) collecting and analyzing representative composite samples of the material initially determined to comprise clean fill; and (f) and based on these steps, making a final determination that the excavated material comprises clean fill if laboratory analysis following the series of physical observations and field screening tests confirms that the material has not been impacted by a release of a substance.

Although the invention has been described with reference to a particular arrangement of parts, processes, features and the like, these are not intended to exhaust all possible arrangements or features, and indeed many other modifications and variations will be ascertainable to those of skill in the art.

What is claimed is:

1. A method for the determination of whether excess excavated material can be considered to comprise clean fill, said method comprising the steps of:
(a) receiving a load of soil removed from a location and taking a representative sample thereof;
(b) performing a location-based analysis of the soil based at least in part on the location from which the soil was removed;
(c) performing a series of physical observations of the soil;
(d) performing field screening tests on the representative sample of the soil;
(e) making an initial determination that the soil comprises clean fill if results of the location-based analysis, results of the series of physical observations and results of the field screening tests are all indicative of no contamination; and
(f) if the initial determination is that the soil comprises clean fill, then:
(i) combining the load of soil with other loads of soil that have also been initially determined to be clean fill to define a composite load;
(ii) combining the representative samples of all loads of soil that have been combined to define a composite sample;
(iii) subjecting the composite sample to laboratory analysis; and
(iv) making a final determination that all of the loads of soil defining the composite load comprise clean fill if results of the laboratory analysis fall within acceptable parameters.

2. The method of claim 1 wherein the step of performing a location-based analysis comprises the step of comparing the location from which the soil was removed to at least one public environmental record of known or potentially contaminated sites.

3. The method of claim 1 wherein the step of performing a location-based analysis comprises the step of determining whether there have been any in-field observations indicative of a potential release at the location.

4. The method of claim 3 wherein the step of determining whether there have been any in-field observations indicative of a potential release at the location comprises the step of interviewing a driver of a truck used to transport the load of soil.

5. The method of claim 1 wherein the series of physical observations comprise at least one of visual observations and olfactory observations.

6. The method of claim 5 wherein the series of physical observations comprise visual observations and wherein the visual observations are considered to be indicative of contamination if staining and/or debris is observed.

7. The method of claim 5 wherein the series of physical observations comprise olfactory observations and wherein the olfactory observations are considered to be indicative of contamination if chemical, petroleum or otherwise offensive odors are observed.

8. The method of claim 1 wherein the series of physical observations are performed by at least two different persons.

9. The method of claim 1 wherein the field screening tests employ a photoionization detector.

10. The method of claim 9 wherein the field screening tests are considered to be indicative of contamination if the photoionization detector indicates a reading of greater than 100 ppm.

11. The method of claim 1 wherein the laboratory analysis comprises tests for at least the following: polychlorinated biphenyls (PCBs) and arsenic, barium, cadmium, chromium, lead, mercury, selenium and silver (total RCRA 8 metals).

12. The method of claim 1 further comprising the step of:
(g) making a determination that the soil comprises contaminated materials if results of at least one of the location-based analysis, the series of physical observations and the field screening tests are indicative of contamination.

13. The method of claim 1 wherein, if the initial determination is that the soil comprises clean fill, then step (f) further comprises the step of:
(v) making a final determination that all of the loads of soil defining the composite load comprise contaminated materials if results of the laboratory analysis fall outside acceptable parameters.

14. A method for the determination of whether excess excavated material can be considered to comprise clean fill, said method comprising the steps of:
(a) receiving a load of soil removed from a location and taking a representative sample thereof;
(b) performing a location-based analysis of the soil based at least in part on the location from which the soil was removed by comparing the location from which the soil was removed to at least one public environmental record of known or potentially contaminated sites and by determining whether there have been any in-field observations indicative of a potential release at the location;
(c) performing a series of physical observations of the soil, wherein the series of physical observations comprise at least one of visual observations and olfactory observations;
(d) employing a photoionization detector to perform field screening tests on the representative sample of the soil;
(e) making an initial determination that the soil comprises clean fill if results of the location-based analysis, results of the series of physical observations and results of the field screening tests are all indicative of no contamination; and
(f) if the initial determination is that the soil comprises clean fill, then:
(i) combining the load of soil with other loads of soil that have also been initially determined to be clean fill to define a composite load;
(ii) combining the representative samples of all loads of soil that have been combined to define a composite sample;
(iii) subjecting the composite sample to laboratory analysis; and
(iv) making a final determination that all of the loads of soil defining the composite load comprise clean fill if results of the laboratory analysis fall within acceptable parameters.

15. The method of claim 14 wherein the step of determining whether there have been any in-field observations indicative of a potential release at the location comprises the step of interviewing a driver of a truck used to transport the load of soil.

16. The method of claim 14 wherein the series of physical observations comprise visual observations and wherein the visual observations are considered to be indicative of contamination if staining and/or debris is observed.

17. The method of claim 14 wherein the series of physical observations comprise olfactory observations and wherein the olfactory observations are considered to be indicative of contamination if chemical, petroleum or otherwise offensive odors are observed.

18. The method of claim 14 wherein the series of physical observations are performed by at least two different persons.

19. The method of claim 14 wherein the field screening tests are considered to be indicative of contamination if the photoionization detector indicates a reading of greater than 100 ppm.

20. The method of claim 14 wherein the laboratory analysis comprises tests for at least the following: polychlorinated biphenyls (PCBs) and arsenic, barium, cadmium, chromium, lead, mercury, selenium and silver (total RCRA 8 metals).

21. The method of claim 14 further comprising the step of:
(g) making a determination that the soil comprises contaminated materials if results of at least one of the location-based analysis, the series of physical observations and the field screening tests are indicative of contamination.

22. The method of claim 14 wherein, if the initial determination is that the soil comprises clean fill, then step (f) further comprises the step of:
(v) making a final determination that all of the loads of soil defining the composite load comprise contaminated materials if results of the laboratory analysis fall outside acceptable parameters.
